(19) United States
(12) Patent Application Publication
(10) Pub. No.: US 2007/0009983 A1
(43) Pub. Date: Jan. 11, 2007

(54) Pyridine nucleotide dehydrogenase based biosensor electrodes

(76) Inventor: Judy Hirst, Cambridge (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/551,144

(22) PCT Filed: Mar. 25, 2004
(86) PCT No.: PCT/GB04/01314

§ 371 Date: Sep. 14, 2006

(30) Foreign Application Priority Data
Mar. 28, 2003 (GB) 0307233.7

(51) Int. Cl. C12Q 1/30; C12Q 1/00

(52) U.S. Cl. 435/27; 205/777.5

(57) ABSTRACT

The invention provides an electrode for the electrochemically reversible interconversion of the oxidised and reduced versions of a pyridine nucleotide comprising: an electrically conducting surface; an isolated pyridine nucleotide dehydrogenase module of an enzyme; wherein said isolated pyridine nucleotide dehydrogenase module is applied to the electrically conducting surface. The isolated pyridine nucleotide dehydrogenase module may be the Iλ subcomplex of bovine mitochondrial NADH: ubiquinone oxidoreductase. Electrochemical cells comprising these electrodes are also provided by the invention.

### BACKGROUND OF THE INVENTION

Numerous studies have been performed in recent years into enzymatic oxidation and reduction reactions employing pyridine cofactors such as NADH (nicotinamide adenine dinucleotide). In order to exploit these reactions in vitro, it is necessary to recycle the NADH product of the enzymatic reaction and regenerate the NAD⁺ reactant. One highly controllable way of recycling the NADH component is via electrochemical oxidation. This also has the advantage that the monitoring of the current flowing in the electrolytic cell offers a convenient way of monitoring the reaction progress as a larger concentration of NADH in the electrolyte will result in a larger oxidation current.

It has been previously shown that it is possible to directly oxidise NADH to NAD⁺ on the surface of an electrode, for example by Gorton [J. Chem. Soc., Faraday Trans. 1, (1986) 82, 1245-1258]. However, the major problem with this reaction is the requirement for a large overpotential to perform the reaction. This results in the formation of radical NAD• species which combine on the electrode surface to form (NAD)₂ dimers, as shown by Jaegfeldt [Bioelectrochem. Bioenerg. 8 (1981) 355] which contaminate the surface of the electrode and decrease the reactant concentration in the electrolyte. A further disadvantage of using a high overpotential is that other non-specific reagents present in the electrolyte are also oxidised at the electrode surface, resulting in further contamination, and in measured oxidation currents not being proportional to the concentration of NADH. These oxidation current measurements then require correction to allow for oxidation of contaminant species.

Work has been performed which is aimed at reducing the overpotential required to drive the NADH oxidation and hence avoid these undesirable effects. One method involves the use of electron transfer agents which can be coated onto the electrode to facilitate electron transfer between the NADH and the electrode surface. Compounds such as ortho-quinones, benzoquinones, phenoxazine, phenothiazine and phenazine dyes have all been used to pretreat the surface of the electrode allowing the oxidation of NADH at lower overpotentials. U.S. Pat. No. 5,122,456 describes the platination or palladation of an activated carbon electrode surface in order to facilitate the transfer of electrons between the electrode and the NADH/NAD⁺ redox couple. U.S. Pat. No. 5,866,353 describes the use of diazacyanine mediators to facilitate the electron transfer between the electrode surface and NADH or NADPH to regenerate NAD⁺ or NADP⁺ respectively.

A second method of reducing the overpotential required to oxidise NADH at the electrode has been developed. This involves the use of an enzyme immobilised on the surface of the electrode which mediates the oxidation of NADH to NAD⁺. U.S. Pat. No. 5,538,867 describes the immobilisation of cytoplasmic hydrogenases from a range of bacteria on the surface of an electrode to lower the overpotential required to drive NAD⁺ reduction. U.S. Pat. No. 5,543,326 describes the modification of redox enzymes to enhance their binding to an electrode surface.

It is known in the art that NAD⁺ and NADH are the substrates for over 350 dehydrogenase enzymes thus a method of performing redox electrochemistry in both a controllable and reversible manner will open up a huge range of potential amperometric enzyme electrodes for a range of different substrates. Direct electron transfer has proven to be an excellent way of studying enzyme mediated redox catalysis on the surface of electrodes. For example, direct electron transfer and high catalytic rates have been demonstrated by Pershad et al. [Biochemistry 38 (1999) 8992-8999] for both hydrogen oxidation and proton reduction using Chromatium vinosum hydrogenase immobilised on an electrode surface.

A problem remaining in all of the work performed on the electrochemical interconversion of oxidised and reduced forms of pyridine nucleotides is that the reaction still requires a significant overpotential to proceed. None of the prior methods for interconversion of pyridine nucleotides provide a truly electrochemically reversible method of doing so.

### BRIEF DESCRIPTION OF FIGURES

FIG. 1—Shows a schematic of the Iλ subcomplex as a part of complex I membrane bound and catalysing NADH oxidation, and isolated and adsorbed onto an electrode, catalysing the reversible interconversion of NADH and NAD⁺.

FIG. 2—Shows a protein film voltammogram of the Iλ subcomplex adsorbed onto a graphite electrode in the presence of NADH and NAD⁺.

FIG. 3—Shows a schematic of the Iλ subcomplex catalysing NADH reduction in the presence of a second enzyme which uses NAD⁺ as a cofactor for converting a substrate to a product.

FIG. 4—Shows an SDS PAGE gel showing fractionation of the individual subunits from which the Iλ subcomplex is composed.

FIG. 5—Shows a plot of isosbestic points (E_{NAD+/NADH}) vs. pH for the Iλ subcomplex adsorbed onto a graphite electrode and in the presence of NADH and NAD⁺.

### SUMMARY OF THE INVENTION

The aim of the present invention is to provide a fully electrochemically reversible method of driving a pyridine nucleotide redox couple in either an oxidative or reductive fashion through immobilisation of an isolated pyridine nucleotide dehydrogenase module of an enzyme on an electrode surface which is then brought into contact with an electrolyte containing reduced and/or oxidised forms of the pyridine nucleotide.

We have surprisingly found that the use of an isolated dehydrogenase module derived from a holoenzyme can, when applied to an electrode, mediate the interconversion of oxidised and reduced forms of a pyridine nucleotide with little or no overpotential required to drive the reaction in either direction. This is entirely unexpected in the light of the prior art in this area.

Accordingly the present invention provides an electrode for the electrochemically reversible interconversion of the oxidised and reduced versions of a pyridine nucleotide comprising: an electrically conducting surface; and an isolated pyridine nucleotide dehydrogenase module of an enzyme; wherein said isolated pyridine nucleotide dehydrogenase module is applied to the electrically conducting surface. In a preferred embodiment, the isolated pyridine nucleotide dehydrogenase module is derived from one of the enzymes selected from the group comprising: NADH:quinone oxidoreductase (complex I, EC 1.6.5.3 or EC 1.6.99.3), sodium translocating NADH:quinone oxidoreductases (Na⁺-NQR), soluble cytoplasmic hydrogenases (for example EC 1.12.1.2) and soluble dehydrogenases. More preferably, the isolated pyridine nucleotide dehydrogenase module is the Iλ subcomplex of bovine mitochondrial NADH:ubiquinone oxidoreductase. Also provided are electrodes in which the isolated pyridine nucleotide dehydrogenase module of an enzyme may be chemically or physically modified such that the pyridine nucleotide dehydrogenase activity is retained.

According to the invention, the enzyme may be applied to the surface of the electrode by a method selected from the group comprising: physisorbtion; ionic interaction; chemisorbtion; hydrophobic interaction and binding in a polymer matrix.

In another embodiment, recombinant DNA methods known in the art may be used to express the subunits of said isolated pyridine nucleotide dehydrogenase module of an enzyme in a host cell which may be assembled in the host cell or recovered from a host cell and assembled in vitro.

Also described are electrodes wherein the electrically conducting surface of the electrode may be made from a material selected from the group consisting of: carbon; gold; silver; platinum; palladium; tungsten; iridium and well doped semi-conductor electrodes such as titanium oxide, indium oxide, tin oxide or diamond. In a more preferable embodiment the electrically conducting surface is a carbon material which may be chosen from the group comprising: glassy carbon; highly ordered pyrolytic graphite (HOPG); edge oriented pyrolytic graphite; graphite.

The invention further comprises an electrochemical cell comprising: a working electrode of the invention; a reference electrode; one or more electrolytes and optionally an auxiliary electrode. The reference electrode may be any commonly used reference electrode for example a standard calomel electrode or a silver/silver chloride electrode.

The electrolyte of the invention comprises one or more buffers selected from the group comprising any commonly used buffers for example sodium acetate, potassium phosphate, MES, HEPES, TAPS, and may optionally comprise a body fluid sample.

The invention also provides an electrochemical cell of the invention further comprising: a substrate for a second enzyme; and a second enzyme capable of converting the substrate into a product; wherein said second enzyme of the invention may use a pyridine nucleotide as a cofactor. In a preferred embodiment, the second enzyme may be a dehydrogenase. More preferably, the second enzyme of the invention may be selected from the group comprising EC numbers: 1.1.1; 1.2.1; 1.3.1; 1.4.1; 1.5.1; 1.7.1; 1.8.1; 1.10.1; 1.11.1.1; 1.11.1.2; 1.12.1; 1.14.12; 1.14.13; 1.16.1; 1.17.1; 1.20.1.

Furthermore the invention discloses a method for effecting the electrochemically reversible interconversion of the oxidised and reduced forms of a pyridine nucleotide in an electrolyte containing the oxidised form and/or the reduced form of the pyridine nucleotide said method comprising applying a potential between the working electrode and the reference electrode of an electrolytic cell of the invention. Preferably the potential applied between the working electrode and the reference electrode is such that the current flowing through the electrochemical cell is proportional to the concentration of either the oxidised or the reduced form of the pyridine nucleotide present in the electrolyte.

In a preferred embodiment of the method of the invention, said potential applied between the working electrode and the reference electrode may be an oxidative or reductive potential converting the reduced or oxidised form of the pyridine nucleotide into the corresponding oxidised or reduced form. In these cases the method may further comprise: monitoring the current flowing through the electrochemical cell; and relating said current to the corresponding concentration of reduced or oxidised pyridine nucleotide respectively using equations well known in the art.

The present invention further provides a method of altering the relative concentrations of oxidised and reduced forms of a pyridine nucleotide in the solvent of an electrochemical cell of the invention containing the oxidised and/or reduced forms of said pyridine nucleotide wherein said method involves applying a potential difference between the working electrode and the reference electrode of the electrochemical cell.

Similarly, the present invention comprises a method of altering the rate of conversion of a substrate to a product in the solvent of an electrochemical cell of the invention comprising a second enzyme which mediates the conversion of substrate to product and uses the pyridine nucleotide of the electrochemical cell as a cofactor, said method comprising, applying a potential difference between the working electrode and the reference electrode of the electrochemical cell.

The present invention also provides a method for measuring the concentration of a substrate in an electrolyte of an electrochemical cell of the invention wherein said method comprises: applying a potential difference between the working electrode and the reference electrode; monitoring the current flowing through the electrochemical cell; and relating said current to the concentration of the substrate.

In preferred embodiments, the pyridine nucleotide of the invention may be NADH, NAD⁺, NADPH or NADP⁺.

### DETAILED DESCRIPTION OF THE PREFERRED EMBODIMENTS

Throughout the specification, the term “isolated pyridine nucleotide dehydrogenase module” is used to refer to a subcomplex, comprising one or more subunits, of an enzyme which modulates the interconversion of the oxidised and reduced forms of a pyridine nucleotide. Many enzymes can be described as comprising modular units in which certain processes are localised. Many of these type of enzymes have more than one active site at which oxidation and reduction of cofactors takes place. The redox processes at these different active sites may be electrochemically coupled. Hence the oxidation of, for example, NADH in one subunit of such an enzyme will produce free electrons which are transferred via internal electron transfer pathways to other subunits of the enzyme. In a number of enzymes, Fe—S clusters form the basis of these electron transfer pathways. The electrons are then used at the electrochemically coupled active site to reduce other enzyme cofactor species. This can be thought of conceptually in electrochemical terms as two half-cell reactions which are coupled such that the electrons from one redox half-reaction are utilised in another spatially separated half-reaction within the enzyme. In enzymes which catalyse the oxidation of pyridine nucleotides, the oxidative activity is often localised into a module of the enzyme comprising one or more protein subunits. The active site within the pyridine nucleotide dehydrogenase module is generally linked to other regions of the enzyme by electron transfer pathways.

This invention is characterised by the uncoupling of the module of the enzyme in which the pyridine nucleotide redox half-reaction occurs from other enzymatic redox half-reactions. The deposition of the uncoupled module of the enzyme onto an electrode surface allows the electrode to take the place of the other half-reactions in the electron transfer processes. This allows direct electron transfer between the pyridine nucleotide redox couple and the electrode surface, via the electron transfer pathways, without interference from other redox reactions.

The subcomplex of the invention may consist of the extrinsic arm of a transmembrane protein such as the NADH:quinone oxidoreductases (complex I, EC 1.6.5.3 and 1.6.99.3). The pyridine nucleotide molecule preferably refers to NADH, NAD⁺, NADPH or NADP⁺. In a preferred embodiment, the isolated pyridine nucleotide dehydrogenase module consists of the Iλ subcomplex of NADH:ubiquinone oxidoreductase from bovine heart mitochondria. A technical advantage is that the enzyme active site is specific for the substrates NADH and NAD⁺. It will not react with other molecules in solution and therefore a highly specific electrode can be formed at which side reactions are negligible.

Suitable pyridine nucleotide dehydrogenase enzymes from which the isolated modules of the invention can be derived include: NADH:quinone oxidoreductases (EC 1.6.5.3 and 1.6.99.3) which can be derived from both prokaryotic and eukaryotic sources; sodium translocating NADH:quinone oxidoreductases (Na⁺-NQR) which can be typified by the enzymes from Vibrio alginolyticus and Vibrio cholerae; and soluble dehydrogenases or soluble cytoplasmic hydrogenases typified by that from Ralstonia eutrophus (EC 1.12.1.2).

Enzymes of this type can be broken into their composite modular units by methods known in the art utilising a wide variety of disruption agents. The disruption agents used in the present invention may be any chaotropic salts or detergents known in the art for example dodecyltrimethyl-ammonium bromide (DTAB), lauryldimethylamine oxide (LDAO) or sodium perchlorate.

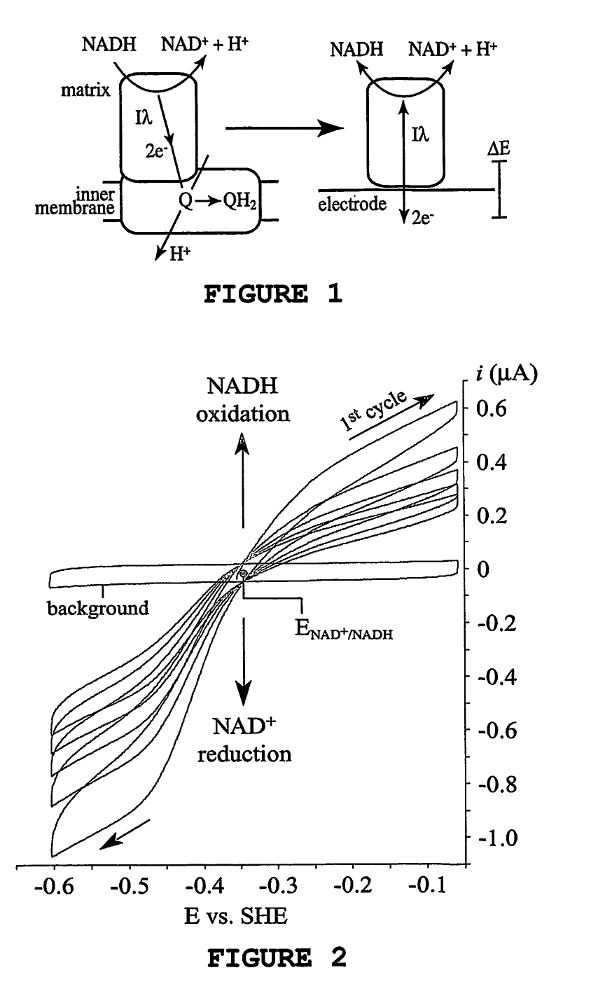

The NADH:ubiquinone oxidoreductase from bovine heart mitochondria used in the invention consists of an assembly of 46 different protein subunits. Following disruption, the NADH dehydrogenase module is isolated in solution by centrifugation on a linear sucrose gradient followed by concentration and gel filtration as is standard in the art and described in example 1. The isolated Iλ NADH dehydrogenase module has a structure which consists of 14 or 15 protein subunits, one flavin mononucleotide (FMN) and a chain of an estimated eight Fe—S clusters along which electrons can be transferred. FIG. 1 shows a schematic of the relationship between the NADH:ubiquinone oxidoreductase and the NADH dehydrogenase module. FIG. 4 shows an SDS PAGE gel showing the number of protein subunits which make up the Iλ subcomplex.

Smaller subcomplexes of this enzyme are known which are also useful in the present invention. An example of this is the flavoprotein subcomplex derived from NADH:ubiquinone oxidoreductase (complex I) from bovine heart mitochondria which consists of three protein subunits, one FMN and two Fe—S clusters. The flavoprotein subcomplex comprises the 51 kDa, 24 kDa and 10 kDa subunits of the Iλ subcomplex. The flavoprotein subcomplex may be isolated by standard methods well known in the art [Galante et al. Archives of Biochemistry and Biophysics 192 (1979) 559-568] using sodium perchlorate as the chaotropic salt.

In addition to the three subunit flavoprotein subcomplex from bovine NADH:ubiquinone oxidoreductase (complex I), an over-expressed enzyme comprising the prokaryotic (E. coli) homologues to the 75 kDa, 51 kDa and 24 kDa Iλ subunits may also describe a useful embodiment of the enzyme for adsorption to the working electrode of the present invention. This three protein subcomplex may be isolated by standard methods well known in the art [Braun et al. Biochemistry 37 (1998) 1861-1897]. Equivalent subcomplexes containing the NADH binding site from other organisms may also be useful in the present invention.

Recombinant DNA methods may also be used to express the subunits of the module in a host cell, which may be assembled in the host cell or recovered from a host cell and assembled in vitro. Recombinant DNA methods may be particularly relevant for expression of the small flavoprotein subcomplexes.

The term “overpotential” is used to refer to the potential required over and above the equilibrium electrode potential in order to induce a current to flow between the working electrode and a second electrode in an electrolytic cell. In a voltammogram with current on the vertical axis and potential on the horizontal axis, the overpotential of a system can be visualised as the substantially horizontal section of the system response in which there is little or no current flowing despite a variation in potential away from the equilibrium potential.

The term “electrochemically reversible” is used to refer to a reaction at the working electrode of an electrochemical cell in which little or no applied overpotential is required to induce a current flow in the cell. In a voltammogram with current on the vertical axis and potential on the horizontal axis, this is visually displayed as a system response characterised by the absence of a substantially horizontal part of the response in which there is little or no current flowing despite a variation in potential away from the equilibrium potential.

Electrochemical cells for use in accordance with the invention may be constructed according to conventional methods known as such in the art. For example, U.S. Pat. No. 5,866,353 describes construction of an electrolytic cell for evaluation of mediator compounds bound to an electrode surface. U.S. Pat. No. 5,122,456 discloses apparatus for determining the amount of NADH in solution through contacting the solution with an activated carbon electrode.

Typically, the electrochemical cell may be constructed comprising a working electrode immersed in an electrolyte solution. A reference electrode is immersed in an electrolyte which may or may not be the same as that in which the working electrode is immersed. The working electrode is in electrical contact with the reference electrode either by being immersed in the same electrolyte solution in the same vessel or by being immersed in an electrolyte which is electrically linked to the reference electrode electrolyte by a salt-bridge or a semi-permeable membrane.

Preferably the electrochemical cell further comprises an auxiliary electrode immersed in the electrolyte. The auxiliary electrode is in electrical contact with the working electrode either by being immersed in the same electrolyte solution in the same vessel or by being immersed in an electrolyte which is electrically linked to the reference electrode electrolyte by a salt-bridge or a semi-permeable membrane.

The salt-bridge or semi-permeable membrane is in contact with both electrolyte solutions and is permeable to charge carrying species but does not allow direct mixing of the electrolytes.

Working electrodes to which the isolated pyridine nucleotide dehydrogenase module may be applied can be selected from electrically conducting materials commonly used in the art for the construction of electrolytic cells. For example P. N. Bartlett et al. [Bioelectrochemistry, 56 (2002) 117-122] describes the use of carbon microelectrodes for the evaluation of electron transfer mediators for NADH oxidation. In addition U.S. Pat. No. 6,340,597 describes the manufacture of biosensor electrodes by formation of composite electrodes from electro-conducting materials, chemical mediator compounds and a solid binding compound.

A working electrode may be constructed from an electrically conducting material including carbon, gold, silver, platinum, palladium, tungsten, iridium and well doped semiconductor electrodes such as titanium dioxide, indium oxide, tin oxide, or diamond.

In a preferred embodiment, the working electrode comprises a carbon component the surface of which is exposed to the electrolyte solution. The carbon component may be made from a material selected from the group comprising: graphite, highly-ordered pyrolytic graphite (HOPG), edge oriented pyrolytic graphite, and glassy carbon. The carbon component may be a disc shape of diameter between 0 and 100 mm, preferably between 0.1 and 20 mm, more preferably between 1 and 5 mm, and typically about 3 mm. This disc may be embedded centrally into the end of a cylinder of electrically insulating material such that one face of the carbon disc is exposed.

The isolated pyridine nucleotide dehydrogenase module is applied to the surface of the working electrode which it uses as an electron acceptor or donor for oxidation of the pyridine nucleotide (at high potentials) or reduction of the pyridine nucleotide (at low potentials). In a preferred embodiment, the isolated pyridine nucleotide dehydrogenase module is in aqueous solution at between 1 μM and 10 mM, preferably between 5 μM and 100 μM, more preferably between 20 μM and 50 μM and even more preferably about 30 μM. This solution may be physically applied to the electrode surface at a coverage of preferably between 0 and 2 μl/mm², more preferably between 0 and 1 μl/mm², more preferably between 0.1 and 0.5 μl/mm², even more preferably about 0.1 μl/mm² after which the solvent is allowed to evaporate.

The isolated pyridine nucleotide dehydrogenase module may be applied to the surface of the working electrode by simple physical application such as immersing the electrode in a solution of the isolated pyridine nucleotide dehydrogenase module or applying a solution of said module to the electrode surface and allowing the solvent to evaporate. This results in the physisorbtion of the isolated pyridine nucleotide dehydrogenase module.

In another embodiment, the isolated pyridine nucleotide dehydrogenase module may be modified in such a way that the dehydrogenase activity is retained but the chemical or physical interaction with the surface of the working electrode is increased. Methods of modifying enzymes are well known in the art, for example U.S. Pat. No. 5,543,326 describes a range of enzyme modifications leading to an increased adhesion of the enzyme to an electrode surface.

In use, the working electrode may be rotated to ensure that a continuous supply of reactants to the electrode/electrolyte interface is maintained. The speed of rotation of the working electrode may be between 0 and 5000 revolutions per minute (rpm), preferably between 100 and 3000 rpm, or between 800 and 1200 rpm, even more preferably about 1000 rpm.

In a preferred embodiment a laminar flow of electrolyte is maintained across the end of the electrode cylinder.

In use, the working electrode may be operated substantially in the absence of oxygen either as an atmosphere or dissolved in the electrolyte. To achieve this, the electrode may be operated under an atmosphere of inert gas and using a degassed electrolyte or one purged with inert gas. The inert gas may be any one of helium, argon, nitrogen, neon, krypton, xenon. Preferably the concentration of oxygen in the operating atmosphere is less than 50 ppm, more preferably less than 20 ppm, even more preferably less than 5 ppm. This can be achieved by using the electrolytic cell in an anaerobic glovebox, which is standard apparatus in the art.

The reference electrode is a standard reference electrode a number of which are well known in the art such as a standard calomel electrode or a silver/silver chloride electrode. Preferably the reference electrode is a standard calomel electrode.

The auxiliary electrode comprises an electrically conducting component the surface of which is exposed to the electrolyte solution. The auxiliary electrode may be made from an electrically conducting material selected from the group consisting of: platinum; palladium; gold; silver; iridium; tungsten; nickel and carbon. Preferably the auxiliary electrode is a platinum wire.

In the preferred three electrode electrolytic cell, the electrodes are electrically linked to a potentiostat [e.g. an Autolab Electrochemical Analyzer (EcoChemie, The Netherlands)], which is apparatus well know in the art, in such a way that a potential can be applied and measured between the working electrode and the reference electrode and a current can flow and be measured between the working electrode and the auxiliary electrode.

In a two electrode system, the electrodes are similarly electrically linked to a potentiostat, in such a way that a potential can be applied and measured between the working electrode and the reference electrode and a current can flow and be measured also between the working electrode and the reference electrode.

The electrolyte solution comprises a liquid containing free charge carriers and optionally one or more pH buffers. In the presence of pH buffers, the solution may be buffered to any pH at which the enzymes of the invention are stable. Preferably the solution is maintained at a pH of between 5 and 10 and more preferably about pH 8. The charge carriers may be selected from the group consisting of: protons; hydroxide ions; halide ions; metal ions; ammonium ions and oxyanions such as nitrate ions; phosphate ions or sulphate ions. The liquid may be water, dimethylsulphoxide (DMSO) or acetonitrile and is preferably water. Suitable pH buffers include any buffer well known in the art and may include sodium acetate, MES, HEPES, TAPS or potassium phosphate. The electrolyte solution may contain any supporting electrolyte salt well known in the art to enhance electrical conductivity of the solution. In a preferred embodiment, the electrolyte solution contains between 10 and 200 mM NaCl as a supporting electrolyte in water. More preferably, the electrolyte solution contains about 100 mM NaCl in water.

In an electrolytic cell, with the isolated pyridine nucleotide dehydrogenase module adsorbed onto the working electrode, the current flowing with the working electrode at a given potential relative to the reference electrode, may be used as a quantitative measure of the amount of oxidised or reduced pyridine nucleotide present in the electrolyte. The current flowing in the electrochemical cell can be related to the concentration of the oxidised or reduced form of the pyridine nucleotide present in the electrolyte through equations which are well know in the art.

Furthermore, a potential applied to the working electrode can be used to drive the pyridine nucleotide redox couple in either an oxidative fashion, through the application of a positive potential relative to the potential of the pyridine nucleotide couple, or a reductive fashion, through the application of a negative potential relative to the pyridine nucleotide couple.

FIG. 2 shows how the current flowing in the electrolytic cell responds to the applied potential as the potential is scanned between two limits. The current flowing can be thought of as the catalytic rate of oxidation or reduction and the potential applied can be thought of as the thermodynamic driving force. As indicated in FIG. 2 scanning in the positive potential direction results in a positive current flowing indicating the oxidation of pyridine nucleotide. Conversely, scanning in the negative potential direction results in a negative current flowing indicating pyridine nucleotide reduction.

In FIG. 2 the waveform described by the current response (with contributions from both oxidation and reduction of the pyridine nucleotide) is a single, continuous sigmoidal wave. The absence of a horizontal part of the voltammogram with current equal to zero, with current on the vertical axis and potential on the horizontal axis shows that the electrocatalytic interconversion of oxidised and reduced forms of the pyridine nucleotide is electrochemically reversible.

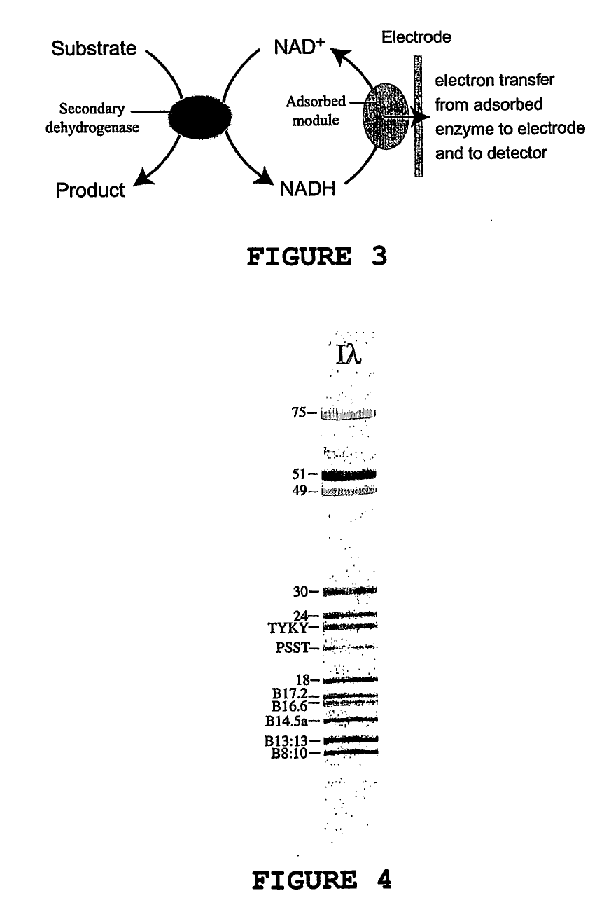

In one embodiment the electrolyte in the electrochemical cell further comprises a second enzyme. This second enzyme uses a pyridine nucleotide as a cofactor in the conversion of a substrate into a product as shown schematically in FIG. 3. Reduced pyridine nucleotide is then converted back to its oxidised form at the working electrode by the immobilised pyridine nucleotide dehydrogenase module. Preferably the conversion of substrate into product at the secondary enzyme is the rate limiting step with respect to the oxidation of the pyridine nucleotide.

In an analogous system, the second enzyme may convert the pyridine nucleotide from its reduced form to its oxidised form in its conversion of a substrate into a product. In this case oxidised pyridine nucleotide is then converted back to its reduced form at the working electrode by the immobilised pyridine nucleotide dehydrogenase module.

The second enzyme may in principle be any dehydrogenase known in the art which uses a pyridine nucleotide as a cofactor. The second enzyme may be selected from the group comprising enzymes with EC numbers: 1.1.1; 1.2.1; 1.3.1; 1.4.1; 1.5.1; 1.7.1; 1.8.1; 1.10.1; 1.11.1.1; 1.11.1.2; 1.12.1; 1.14.12; 1.14.13; 1.16.1; 1.17.1; 1.20.1.

Possible substrates for these second enzymes are well defined and described in detail in the art. The range of possible substrates for a given enzyme may be determined by reference to EC number databases commonly used by those skilled in the art.

In the embodiment comprising a second enzyme, the electrical current measured between the working electrode and the auxiliary electrode may be directly related to the rate of conversion of the substrate to product at the second enzyme. As such, this embodiment provides a method of detecting and quantifying a variety of substrates in solution through careful selection of the second enzyme.

It will be apparent that electrodes of the present invention may be used in biosensor applications. Such an embodiment may provide a method for the amperometric determination of the concentration of an analyte in a biological fluid sample. This biological fluid sample may be a body fluid such as blood, plasma or urine. The electrodes of the present invention are particularly suited to the analysis of biological fluids due to the absence of a significant required overpotential. This results in the minimised oxidation of other fluid components, such as vitamin C, uric acid, or acetaminophen. This reduces the error in amperometric measurements from fluid samples due to the oxidation of other fluid components. Errors of this type occur especially in systems requiring a high overpotential.

Biosensor applications of the present electrode invention may include the direct detection of pyridine nucleotides and measurement of their concentration in solution. Further embodiments may also include the use of the second enzyme system of the invention in the amperometric measurement of concentrations of other biological fluid analytes by utilising a variety of possible second enzymes. Biological fluid analytes of these embodiments may include, blood ketones, hormones such as testosterone, steroids, amino-acids notably phenylalanine, lactate, sugars, alcohols, and pharmaceuticals and their metabolites. In the detection of these various analytes, a second enzyme may be chosen which converts the analyte into a product with concomitant transformation of a pyridine nucleotide from its oxidised form to its reduced form (or alternatively from its reduced form to its oxidised form). The original form of the pyridine nucleotide is then regenerated at the isolated pyridine nucleotide dehydrogenase module adsorbed onto the electrode surface. The current flowing in the electrolytic cell can then be related to the concentration of one form of the pyridine nucleotide in solution and hence to the concentration of the second enzyme substrate in the electrolyte.

A further application of the embodiment comprising a second enzyme is to drive a reaction in solution. In this use of the electrode, a potential is applied to the electrode to which the isolated pyridine nucleotide dehydrogenase module is adsorbed, such that the pyridine nucleotide in solution is transformed from its reduced to its oxidised form by the adsorbed module. The second enzyme in the system may be selected such that it uses the oxidised form of the pyridine nucleotide as a cofactor in transforming a substrate to a product. The increased concentration of the oxidised pyridine nucleotide, and the lack of the reduced pyridine nucleotide, in solution then drives the second enzyme to convert substrates to products.

In an analogous manner, the reverse reaction also represents a useful embodiment of this invention. In this case, a pyridine nucleotide in solution is transformed from its oxidised to its reduced form by the adsorbed module on application of a potential to the electrode to which the isolated pyridine nucleotide dehydrogenase module is adsorbed. The second enzyme in the system is then selected such that it uses the reduced form of the pyridine nucleotide as a cofactor in transforming a substrate to a product. The increased concentration of the reduced pyridine nucleotide, and the lack of the oxidised pyridine nucleotide, in solution then drives the second enzyme to convert substrates to products.

Through the careful selection of the second enzyme, a wide range of enzyme mediated reactions can be controllably driven in solution to form useful or desirable products. In this way, the control of the potential applied to the electrode results in the control of the rate of reaction at the second enzyme, i.e. the rate of formation of products.

Using this “driveable” second enzyme embodiment of the invention, provides access not only to a range of desirable products some of which may only be accessible via enzymatic reactions, but also to a controllable method of making these products.

Examples of applications using this “driveable” second enzyme embodiment of the invention include the synthesis of optically pure compounds which may have uses in pharmaceutical or food additive industries. Embodiments of this type may include the stereoselective synthesis of amino acids from α-keto carboxylic acids in the presence of an ammonium salt and an amino acid dehydrogenase. Reduction of α-keto acids or oxidation of alcohols to give hydroxy acids mediated by either the forward or reverse action of dehydrogenase enzymes may also represent a useful embodiment of this invention.

In addition, reactions of this type may be used to produce alcohols, aldehydes or ketones which are commonly utilised in the chemical industry and in the manufacture of perfumes, flavourings and food additives. Examples of reactions of this type include: the reduction of ketones or aldehydes to alcohols, and the corresponding reverse reactions, using alcohols, and the corresponding reverse reactions, using alcohol dehydrogenases; the conversion of sugars into alcohols using dehydrogenase enzymes; and the transformation of diols into chiral lactones using alcohol dehydrogenases.

The functionalisation of steroids can also be performed enzymatically in the presence of a pyridine nucleotide and as such represents a possible further embodiment of the present invention. Examples of this type of reaction are typically dehydrogenations utilising hydroxysteroid dehydrogenases.

A huge number of potential applications are envisaged using this second enzyme embodiment of the invention. In all of the examples of this type of embodiment, the adsorbed enzyme module performs a similar task, that of regenerating the oxidised or reduced form of the pyridine nucleotide which is used as a cofactor by the second enzyme in transforming substrates into products. It is therefore only necessary for the adsorbed enzyme to be present in catalytic amounts to effect regeneration of the oxidised or reduced form of the pyridine nucleotide. It is this regeneration of the oxidised or reduced form of the pyridine nucleotide that enables the second enzyme to maintain turnover of substrate to products.

### EXAMPLE 1

### Isolation of the Iλ Module from Bovine NADH:Ubiquinone Oxidoreductase

An aliquot of solution containing ca. 10 mg mL⁻¹ bovine complex I, LDAO, DTAB, potassium phosphate buffer (pH 7.5) and dithiothreitol, was layered onto a linear sucrose gradient. This sucrose gradient was then centrifuged for 18 hours at 200,000 g. The sharp yellow-brown band in the center of the gradient was collected and concentrated, and further purified using a Superose 6 HR 10/30 gel filtration column. Fractions spanning the apex of the symmetrical absorbance peak were pooled and stored in liquid nitrogen.

SDS PAGE of this product demonstrated that this is indeed the subcomplex Iλ. The SDS PAGE gel is shown in FIG. 4 with subunits marked on the left of the gel.

### EXAMPLE 2

### Adsorption of the Isolated Iλ Module onto a Electrode Surface and Subsequent Voltammetry

Protein film voltammetry (PFV) studies were performed on the isolated Iλ subcomplex of the invention.

1 μL of a 30 μM aqueous solution of subcomplex Iλ was applied to the surface of a freshly polished 3 mm diameter pyrolytic graphite edge rotating-disc electrode. When the solvent had evaporated the electrode was placed into a thermostatted all-glass electrochemical cell. An aqueous solvent was added to the electrochemical cell. The solvent contained 0.1M NaCl as a supporting electrolyte and a mixed buffer system to control the pH of the electrolyte solution. The mixed buffer system consisted of 10 mM sodium acetate, MES, HEPES and TAPS salts. The pH was maintained at 7.82. NAD⁺ and NADH (Roche) were re-purified by standard procedures and then added to the electrolyte each to a concentration of 1 mM. A standard calomel reference electrode was used and an auxiliary electrode consisting of a platinum wire was inserted into the electrolyte. The electrodes were connected to an Auto lab Electrochemical Analyzer (EcoChemie). The working electrode was rotated using an EG&G electrode rotator. The entire apparatus was contained in an anaerobic glovebox with an oxygen concentration of less than 2 ppm.

The potential of the electrochemical cell was held at open circuit for 10 s before scanning. PFV was then performed at a scan rate of 10 mVs⁻¹ at a temperature of 20° C. and with an electrode rotation rate of 1000 rpm.

The voltammogram resulting from repeated scanning of the potential between 0 and −0.6V is shown in FIG. 2. All potentials have been corrected to the standard hydrogen electrode scale [Bard, A. J.; Faulkner, L. R. Electrochemical Methods: Fundamentals and Applications; Wiley: N.Y., 2001].

The single, continuous, sigmoidal waveshape of FIG. 2 confirms that both NADH oxidation and NAD⁺ reduction are electrochemically reversible, and do not require any overpotential.

No signals were observed in the absence of subcomplex Iλ.

### EXAMPLE 3

### Electrochemical Reversibility of NADH/NAD⁺ Interconversion Over a Range of pH Values

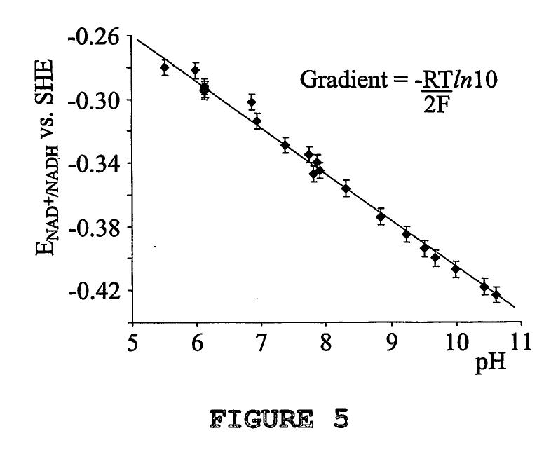

The isosbestic points of FIG. 2 (shown as E_{NAD+/NADH}) denote the potential at which the rate of catalysis in the oxidative direction is equal to that in the reductive direction. In FIG. 2 the concentrations of NADH and NAD⁺ are equal and therefore the isosbestic potential is the reduction potential of NAD⁺ (E_{NAD+/NADH}) A plot of E_{NAD+/NADH} (measured using PFV, from isosbestic potential points) as a function of pH is shown in FIG. 5. E_{NAD+/NADH} varies linearly with pH, over a wide pH range, and conforms to the predictions of the Nernst Equation (solid line in FIG. 5). This indicates that the redox system behaves as predicted theoretically and hence is electrochemically reversible over this wide pH range.

1. An electrode for the electrochemically reversible interconversion of the oxidised and reduced versions of a pyridine nucleotide comprising:
an electrically conducting surface;
an isolated pyridine nucleotide dehydrogenase module of an enzyme;
wherein said isolated pyridine nucleotide dehydrogenase module is applied to the electrically conducting surface.

2. An electrode according to claim 1 wherein said isolated pyridine nucleotide dehydrogenase module of an enzyme is derived from any one of the enzymes selected from the group consisting of: NADH:quinone oxidoreductases (complex I, EC 1.6.5.3 and EC 1.6.99.3); sodium-translocating NADH:quinone oxidoreductases (Na⁺-NQR); soluble cytoplasmic hydrogenases; and soluble dehydrogenases.

3. An electrode according to claim 2 wherein said isolated pyridine nucleotide dehydrogenase module of an enzyme is the Iλ subcomplex of bovine mitochondrial NADH:ubiquinone oxidoreductase.

4. An electrode according to claim 1 wherein said isolated pyridine nucleotide dehydrogenase module of an enzyme is chemically or physically modified such that the pyridine nucleotide dehydrogenase activity is retained.

5. An electrode according to claim 1 wherein said isolated pyridine nucleotide dehydrogenase module of an enzyme is applied to the surface of the electrode by a method selected from the group consisting of: physisorbtion; ionic interaction; chemisorbtion; hydrophobic interaction and binding in a polymer matrix.

6. An electrode according to claim 1 wherein the dehydrogenase module of an enzyme has been obtained by expression of subunits of said module in a recombinant host cell.

7. An electrode according to claim 1 wherein said electrically conducting surface is made from a material selected from the group consisting of: carbon; gold; silver; platinum; palladium; tungsten; iridium and well doped semiconductor electrodes.

8. An electrode according to claim 7 wherein said electrically conducting surface is a carbon material which is chosen from the group consisting of: glassy carbon; highly ordered pyrolytic graphite (HOPG); edge oriented pyrolytic graphite; and graphite.

9. An electrochemical cell comprising:
a working electrode according to claim 1;
a reference electrode; and
one or more electrolytes.

10. An electrochemical cell according to claim 9 further comprising an auxiliary electrode.

11. An electrochemical cell according to claim 9 wherein said reference electrode is a standard reference electrode and is selected from the group consisting of: a standard calomel electrode; and a silver/silver chloride electrode.

12. An electrochemical cell according to claim 9 wherein said electrolyte comprises one or more buffers selected from the group consisting of: sodium acetate; potassium phosphate; MES; HEPES; and TAPS.

13. An electrochemical cell according to claim 9 wherein said electrolyte comprises a body fluid sample.

14. An electrochemical cell according to claim 9 wherein said electrolyte further comprises:
a substrate;
a second enzyme which is effective to convert the substrate into a product;
wherein said second enzyme uses a pyridine nucleotide as a cofactor.

15. An electrochemical cell according to claim 14 wherein said second enzyme is a dehydrogenase enzyme.

16. An electrochemical cell according to claim 15 wherein said second enzyme is selected from the group consisting of enzymes with EC numbers: 1.1.1; 1.2.1; 1.3.1; 1.4.1; 1.5.1; 1.7.1; 1.8.1; 1.10.1; 1.11.1.1; 1.11.1.2; 1.12.1; 1.14.12; 1.14.13; 1.16.1; 1.17.1; 1.20.1.

17. A method for effecting the electrochemically reversible interconversion of the oxidised and reduced forms of a pyridine nucleotide in an electrolyte containing at least one of the oxidised form and the reduced form of the pyridine nucleotide said method comprising applying a potential between the working electrode and the reference electrode of an electrochemical cell wherein said electrochemical cell is as defined in claim 9.

18. A method according to claim 17 wherein the potential applied between the working electrode and the reference electrode is such that the current flowing through the electrochemical cell is proportional to the concentration of either the oxidised or the reduced form of the pyridine nucleotide present in the electrolyte.

19. A method according to claim 17 wherein the reduced form of the pyridine nucleotide is present in the electrolyte and said potential applied between the working electrode and the reference electrode is an oxidative potential converting said reduced form of the pyridine nucleotide into the oxidised form.

20. A method according to claim 19 further comprising:
monitoring the current flowing through the electrochemical cell;
relating said current to the concentration of reduced pyridine nucleotide.

21. A method according to claim 17 or 18 wherein the oxidized form of the pyridine nucleotide is present in the electrolyte and said potential applied between the working electrode and the reference electrode is a reductive potential converting said oxidised form of the pyridine nucleotide into the reduced form.

22. A method according to claim 21 further comprising:
monitoring the current flowing through the electrochemical cell;
relating said current to the concentration of oxidised pyridine nucleotide.

23. A method of altering the relative concentrations of oxidised and reduced forms of a pyridine nucleotide in the electrolyte of an electrochemical cell containing at least one of the oxidised and reduced forms of said pyridine nucleotide wherein said electrochemical cell is as defined in claim 9 and wherein said method involves applying a potential difference between the working electrode and the reference electrode of the electrochemical cell.

24. A method of altering the rate of conversion of a substrate to a product in the electrolyte of an electrochemical cell, wherein said electrochemical cell is as defined in claim 14, comprising applying a potential difference between the working electrode and the reference electrode of the electrochemical cell.

25. A method for measuring the concentration of the substrate in the electrolyte of the electrochemical cell of claim 14 wherein said method comprises:
applying a potential difference between the working electrode and the reference electrode;
monitoring the current flowing through the electrochemical cell; and
relating said current to the concentration of the substrate.

26. A method according to claim 1 wherein said pyridine nucleotide is NADH.

27. A method according to claim 1 wherein said pyridine nucleotide is NADPH.

28. A method according to claim 1 wherein said pyridine nucleotide is NAD⁺.

29. A method according to claim 1 wherein said pyridine nucleotide is NADP⁺.

30. An electrode according to claim 7, wherein said well doped semiconductor electrodes are selected from the group consisting of titanium oxide indium oxide, tin oxide or diamond.
